(19) United States
(12) Patent Application Publication
(10) Pub. No.: US 2012/0004498 A1
(43) Pub. Date: Jan. 5, 2012

(54) DEVICE AND METHOD FOR REDUCING TENSION AND STRESS IN A SUBJECT

(75) Inventors: Dolores MALASPINA, New York, NY (US); Andreas KELLER, New York, NY (US); Daniel ANTONIUS, Buffalo, NY (US)

(73) Assignee: NEW YORK UNIVERSITY, New York, NY (US)

(21) Appl. No.: 13/162,067

(22) Filed: Jun. 16, 2011

Related U.S. Application Data
(60) Provisional application No. 61/355,322, filed on Jun. 16, 2010.

(51) Int. Cl. A61M 21/00

(52) U.S. Cl. 600/27

(57) ABSTRACT

The present invention relates to a sensory device for reducing tension in a subject. The device includes an elastic covering, a filling material inside the elastic covering, an openable sealed reservoir located within the filling, an absorbent material surrounding the reservoir, and a calming odor releasing substance sealed within the reservoir. The present invention also relates to a method of reducing tension in a subject. This method involves providing a subject in need of tension relief with the device of the present invention. The device is manipulated to break open the reservoir and release the calming odor releasing substance. The released calming odor is inhaled in an amount sufficient to lower salivary cortisol levels in the subject and thereby reduce tension in the subject.

This application claims the benefit of U.S. Provisional Patent Application Ser. No. 61/355,322, filed Jun. 16, 2010, which is hereby incorporated by reference in its entirety.

### FIELD OF THE INVENTION

This invention relates to a device and method for reducing tension and stress in a subject.

### BACKGROUND OF THE INVENTION

Patients admitted to psychiatric units in the United States constitute an enormous economic burden on the public sector. Additionally, stress, agitation, aggression, and violence among psychiatric patients adds to the financial costs and represent a serious concern for the treating clinical staff and other staff, as well as peer patients. The tremendous amount of time, effort, and money spent on securing staff and patient safety often makes management and treatment of the agitated and/or aggressive patient difficult. This is particularly evident in the emergency room and inpatient settings where patients often have impaired cognition due to acute psychotic states, substance intoxication/withdrawal, and/or general over-arousal related to the stressful situation.

In these settings, reasoning and conversation are often not effective interventions for violent and aggressive behaviors. Thus, situations rapidly escalate to restraint, seclusion, and involuntary medication for the safety of the patient, other patients, and the staff. Recently, the Joint Commission on Accreditation of Healthcare Organizations (“JCAHO”) stipulated that seclusion and restraint methods to contain challenging patients should be a last resort.

As a result, alternatives to seclusion and restraint in psychiatric settings have become increasingly sought after. Scattered studies in non-psychiatric populations and anecdotal evidence suggest that implementing sensory stimulation approaches can reduce arousal and anxiety. Sensory approaches may be particularly promising because they are flexible and inexpensive. However, sensory methods lack empirical validation in psychiatric settings, as no scientifically rigorous trials have been conducted in, e.g., a busy inner-city hospital's inpatient and emergency psychiatric department.

The present invention is directed to overcoming these and other limitations in the art.

### SUMMARY OF THE INVENTION

One aspect of the present invention relates to a sensory device for reducing tension in a subject. The device includes an elastic covering, a filling material inside the elastic covering, an openable sealed reservoir located within the filling, an absorbent material surrounding the reservoir, and a calming odor releasing substance sealed within the reservoir.

Another aspect of the present invention relates to a method of reducing tension in a subject. This method involves providing a subject in need of tension relief with the device of the present invention. The device is manipulated to break open the reservoir and release the calming odor releasing substance. The released calming odor is inhaled in an amount sufficient to lower salivary cortisol levels in the subject and thereby reduce tension in the subject.

The device of the present invention is advantageous in that it includes an odor releasing substance in an ampoule in the middle of the device. The device can be re-used by replacing the ampoule with the same or a different odor releasing substance. Also, the size of the device is larger than a traditional “tennis ball” size stress ball and is better suited for psychiatric and medical inpatients. The device is made of materials that are soft enough to avoid danger to other patients if the device is thrown to hit another person.

The present invention provides an innovative sensory approach to reducing stress and agitation within inpatient psychiatric settings. Sniffing odors in an experimental situation has been shown to lower levels of the stress hormone cortisol in saliva in healthy individuals. Previous research suggests that sensory engagement and exploration of odors may alone have a calming effect (an effect similar to that of “breathing” as a reducer of stress). In addition to salivary cortisol levels, self-reported mood that was measured before and after odor exposure has been reported to change for a variety of different odors.

The present invention provides an alternative method to de-escalate challenging patients, rendering seclusion and restraint methods a last resort. The device and method of the present invention will inexpensively, easily, and effectively be implemented into hospital and/or psychiatric treatment settings, providing safe and effective stress/agitation reduction, as well as in non-hospital settings such as outpatient clinics, school settings, law enforcement settings and prisons, and the home environment.

The present invention provides alternative methods to de-escalate challenging patients so that seclusion and chemical and physical restraint methods become a last resort. The present invention is, in principal, applicable outside of psychiatric settings and to every environment in which stress and agitation are problematic.

### BRIEF DESCRIPTION OF THE DRAWINGS

FIG. 1 is a cutaway drawing of one embodiment of the sensory device of the present invention.

### DETAILED DESCRIPTION OF THE INVENTION

One aspect of the present invention relates to a sensory device for reducing tension in a subject. The device includes an elastic covering, a filling material inside the elastic covering, an openable sealed reservoir located within the filling, an absorbent material surrounding the reservoir, and a calming odor releasing substance sealed within the reservoir.

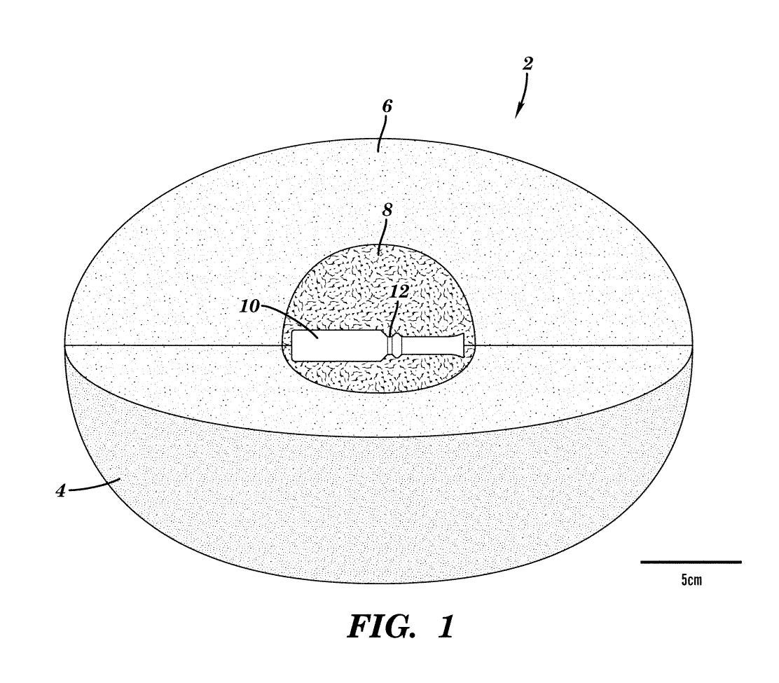

Turning to FIG. 1, sensory device 2 has elastic covering 4 and filling material 6 inside elastic covering 4. Openable sealed reservoir 10 is located within filling material 6. Absorbent material 8 surrounds openable sealed reservoir 10. A calming odor is contained in openable sealed reservoir 10.

Sensory device 2 may be of any shape and/or size. In one embodiment, device 2 has an approximately oval shape, with its length being about two times its diameter. Thus, for example, device 2 may be oval shaped and about 40 cm in length and about 20 cm in diameter. Other shapes and sizes are also contemplated. In one embodiment, device 2 is of a size that is comfortable to hold in the palm of the hand. Whatever its specific construction, device 2 is intended to be light and easily manipulated and squeezed by a subject's hand.

Elastic covering 4 can be constructed of any highly durable elastic material. In one embodiment, elastic covering 4 is a material that is flexible and comfortable to the hand, so that the device can be comfortably held in the palm of the hand. Elastic covering 4 may be constructed of a single type of material or may be a composite material. Suitable materials include natural or synthetic materials, or a mixture of natural and synthetic materials. In one particular embodiment, elastic covering 4 is a nylon and spandex composite, which may include about 84% nylon and about 16% spandex.

In one embodiment, elastic covering 4 has a textured silicone surface. Elastic covering 4 may also be optionally colored or marked with designs or information to, e.g., give an indication of the odor releasing substance contained within device 2.

Filling material 6 inside elastic covering 4 is, according to one embodiment, a porous silicone material or polystyrene foam beans. Any material may be used so long as it gives structure to sensory device 2 and is relatively light-weight and porous.

Openable sealed reservoir 10 is, according to one embodiment, a sealed ampoule containing the calming odor releasing substance. In one embodiment, the sealed ampoule is constructed of plastic material. The ampoule may contain a predetermined breaking-point for facilitating odor release of the odor releasing substance contained within the reservoir.

Suitable calming odor releasing substances may include any substance now known or later discovered to have a calming effect on a subject when inhaled. Natural and synthetic substances are well known in the art. In one embodiment, the odor releasing substance is octyl acetate. For example, device 2 may include about 5 ml of an about 1/100 dilution of octyl acetate. Octyl acetate has scientifically been shown to reduce stress. Octyl acetate has the advantage over natural odor mixtures found in nature in that it can be reliably reproduced and has a defined stimulus that makes receptor-ligand studies and similar endeavors possible.

Other odor releasing substances can also be used in the device of the present invention. For example, other suitable odor releasing substances may include, without limitation, (−)-menthol, (+)-menthol, 1-butanol, 2-butanone, 2-decenal, 2-ethylfenchol, 2-methoxy-4-methylphenol, 4-methylvaleric acid, ambrette, androstadienone, androstenone, anise, banana, bourgeonal, butyl acetate, butyric acid, caproic acid, cedarwood, cineole, cinnamon, cis-3-hexen-1-ol, citral, citronella, decyl aldehyde, diacetyl, diallyl sulfide, diphenyl ether, ethyl vanillin, ethylene brassylate, eugenol, eugenol acetate, eugenol methyl ether, fenchone, fir, galaxolide, geranyl acetate, guaiacol, heptaldehyde, heptyl acetate, hexyl butyrate, isobornyl acetate, isobutyraldehyde, isobutyric acid, isoeugenol, isovaleric acid, jasmine, lime, linalool, methanethiol, methyl salicylate, nonyl aldehyde, nutmeg, octyl aldehyde, orange, pentadecalactone, phenyl acetaldehyde, pyrazine, r-carvone, r-limonene, sandalwood, spearmint, terpineol, terpinyl acetate, undecanal, vanillin, and mixtures thereof.

Absorbent material 8 surrounding openable sealed reservoir 10 is, according to one embodiment, nonwoven polypropylene, although other absorbent materials are well-known in the art and may also be used.

In operation, device 2 is held in, e.g., the palm of a subject's hand. Upon being, e.g., squeezed by the subject's hand, filling material 6 inside elastic covering 4 presses down upon openable sealed reservoir 10 and breaks open openable sealed reservoir 10 to release the calming odor releasing substance contained therein. By this means, calming odors are infused into absorbent material 8, filling material 6 and escape out of elastic covering 4 to be inhaled by the subject near device 2.

Thus, another aspect of the present invention relates to a method of reducing tension in a subject. This method involves providing a subject in need of tension relief with the device of the present invention. The device is manipulated to break open the reservoir and release the calming odor releasing substance. The released calming odor is inhaled in an amount sufficient to lower salivary cortisol levels in the subject and thereby reduce tension in the subject.

In one embodiment of the method of the present invention, the subject is a human subject, although the term “subject” also refers to other non-human animal subjects that may be in need of reduced tension.

Subjects provided pursuant to the method of the present invention are subjects in need of reduced tension and may be expressing symptoms of, e.g., stress, agitation, aggression, and/or violence.

According to the method of the present invention, device 2, e.g., is manipulated to break open sealed reservoir 10. In one embodiment, manipulation occurs by the subject or someone in close proximity to the subject squeezing device 2 in the palm of the hand. The released calming odor is inhaled by the subject in an amount sufficient to lower salivary cortisol levels in the subject and thereby reduce tension in the subject.

Cortisol levels of a subject can be determined through saliva samples taken from the subject. Cortisol is a known biomarker of stress and agitation (S. Cohen, R. C. Kessler, & L. G. Underwood (eds.), Measuring Stress: A Guide for Health and Social Scientists, New York: Oxford University Press (1995), which is hereby incorporated by reference in its entirety). The method of the present invention may reduce salivary cortisol in a subject by about 10%, 11%, 12%, 13%, 14%, 15%, 16%, 17%, 18%, 19% or 20% or more.

### EXAMPLES

The following examples are provided to illustrate embodiments of the present invention but are by no means intended to limit its scope.

### Prophetic Example 1

### Sensory Method Aimed at Reducing Tension

The effectiveness of two different Complementary Alternative Medicine (“CAM”) approaches that will be used in a complementary manner will be compared, in addition to standard conventional care, versus standard conventional care alone in psychiatric units at Bellevue Hospital in New York City. Many CAM methods are soon to be implemented in psychiatric hospitals across the United States, as the JCAHO has stipulated that seclusion and restraint methods to contain agitated patients must become a “last resort.”

In the emergency room and inpatient settings, patients often have impaired cognition due to acute psychiatric states, substance intoxication/withdrawal, and/or over-arousal such that reasoning and conversation are not effective interventions, and situations rapidly escalate to restraint, seclusion, and involuntary medication for the safety of the patient, other patients, and the staff.

Scattered studies in non-psychiatric populations and anecdotal evidence suggest that implementing sensory stimulation approaches can reduce arousal and anxiety, so there is hope that these approaches may have utility in reducing acute agitation and aggressive behavior in psychiatric patients. However, CAM methods lack empirical validation in this setting, as no scientifically rigorous trials have been conducted in, e.g., a busy inner-city hospital's inpatient and emergency psychiatric department.

The device of the present invention will be tested in an inner-city hospital's psychiatric emergency room, and on inpatient civilian and forensic psychiatric units. Through methodologically rigorous study, the effectiveness of treatment will be compared as usual with two novel interventions—tactile-only and tactile-smell—in reducing episodes of restraint, seclusion, and involuntary medication. A preliminary model of the present invention is currently being tested on 150 psychiatric inpatients at Bellevue Hospital Center, New York City.

Other studies will be conducted on the effects of the device of the present invention in acute-, forensic-, out-patients, and in child and adolescent psychiatric populations. Subjects receiving the intervention will also be assessed on clinical measures and a biomarker of arousal and agitation (salivary cortisol) measured before, during, and after the administration. This research team is predominantly comprised of investigators with expertise in forensic and emergency psychiatry, patient-oriented research, and basic olfactory neuroscience.

The studies will compare the effectiveness of different complementary stimuli for self-soothing of agitated, aroused/stressed, and violent psychiatric patients versus standard treatment options.

JCAHO has stipulated that seclusion and restraint methods to contain agitated and problematic psychiatric patients are now to be considered to be a last resort. Complementary and alternative methods to manage these patients are shortly being implemented. Sensory approaches for self soothing may be particularly promising, but these methods lack empirical validation in psychiatric settings.

The framework of all of this research is the well-articulated need to find new and alternative methods to calm aroused, anxious, agitated, and aggressive psychiatric patients. As mentioned above, data is currently being collected on inpatients at Bellevue Hospital in New York City. Data is also expected to be collected on the device and method of the present invention on forensic inpatient psychiatry units, the child and adolescent psychiatry units, and the psychiatric emergency room of Bellevue Hospital.

The aim of this new research is to test the effectiveness of complementary sensory stimuli versus standard care alone for patients who are aroused, anxious, agitated, and aggressive. The sensory stimuli are tactile-only and tactile-smell. The study outcomes are (1) the number of episodes of restraint/seclusion/medication doses in the respective settings, and (2) behavioral ratings and salivary cortisol measures (a biomarker of stress and agitation) in individual patients before, during, and after the interventions. The investigators are experts in forensic and emergency psychiatry, clinical psychiatric research, and olfactory neuroscience.

Aim 1: Recruit 700 Psychiatric Patients at a High Risk for an Escalating Level of Arousal, Anxiety, Agitation, or Aggressive Behavior

Patients will be recruited to participate in the protocol as follows: (1) three hundred cases (N=300) who involuntarily present to the psychiatric emergency room; (2) two hundred cases (N=200) admitted to an adult inpatient psychiatric unit; (3) one hundred cases (N=100) admitted to an inpatient psychiatric forensic unit; and (4) one hundred cases (N=100) admitted to a child or adolescent psychiatric unit.

The sample will include all ages. Sexes will be recruited equally to their proportion in each setting. Note that the Bellevue forensic ward does not house female patients. Demographic and clinical information will be abstracted from chart and clinical interviews with subjects.

Aim 2: Administer the Device for Reducing Arousal, Anxiety, Agitation, or Aggressive Behavior

Participants will be offered the device of the present invention when they are escalated or observed to be escalating. The patients will be told to hold the item and try to “calm down.”

Clinical ratings, patient reported distress, and a salivary sample will be obtained if feasible when they are given the device at 15 minutes after receiving the device and at 30 minutes upon returning the device.

In 3-month-long epochs, the device of the present invention will be tactile-only or tactile-smell or no intervention (treatment as usual) across the study settings to test the following hypotheses:

Hypothesis 1: Cases with the device of the present invention will have significantly fewer episodes of restraint/seclusion/involuntary medication than those who receive the tactile-only device.

Hypothesis 2: Cases with the device of the present invention will have significantly greater declines in clinical ratings and salivary cortisol than those who receive the tactile-only device.

Hypothesis 3: During the study month blocks there will be fewer restraint and seclusion and involuntary medication episodes overall compared to during the non-study (treatment as usual) month blocks in each setting. The ameliorative effect will be greater for the blocks with the tactile-smell device of the present invention than for the blocks with the tactile-only device.

### Prophetic Example 2

### The Challenge and Potential Impact

Opportunity to Advance Research in Reducing Arousal, Anxiety, and Agitation in Psychiatric Patients

The use of restraints for aggressive or assaultive behavior is governed by guidelines of the JCAHO and the Centers for Medicare and Medicaid Services. Guidelines for dealing with the acutely agitated patient have recently been published by an expert consensus panel. All three sources agree that the use of nonphysical forms of behavior management (e.g., verbal intervention) is the appropriate first-line strategy. JCAHO has stipulated that seclusion and restraint methods to control agitated and problematic psychiatric patients are now a last resort.

Thus, alternatives to seclusion and restraint in psychiatric settings have become increasingly important. Sensory stimulation approaches may be particularly promising. These approaches are often implemented under occupational therapy oversight and many such approaches have anecdotally been reported to be successful. Unfortunately, evaluations of the effectiveness of these sensory stimulation approaches are often not published because most of these approaches are not designed as a research study. They do not typically include control groups, attempts to double-blind, or an objective measure of the physiological effects of the treatment.

One may argue that with outcomes such as a 54% reduction of seclusion and restraint rates during implementation of a sensory stimulation approach (Champagne et al., “The Effects of the Use of the Sensory Room in Psychiatry,” www.ot-innovations.com/sensoryrooms.html, which is hereby incorporated by reference in its entirety) no rigorous scientific evaluation is necessary. However, the interpretation of such findings is problematic because the implementation of a sensory stimulation approach to early intervention is usually accompanied by an institution-wide push to reduce seclusion and restraint rates that includes staff training and change of policy and procedure in addition to the sensory approach. It is therefore impossible to transfer reported approaches to systematically improve them or to learn from the reported success-rates something about the mechanisms that mediate the desirable effect.

To rectify this, a line of research is proposed to test the effectiveness of sensory stimulation in reducing clinical ratings and a biomarker of stress and agitation (salivary cortisol) in challenging psychiatric settings (i.e., the psychiatric emergency room and inpatient units at the Bellevue Hospital in New York City).

The focus will be on olfactory and tactile stimuli, since those are the sensory modalities that are most closely connected to social functions, the autonomous nervous system, and to the hormonal regulation of responses and other moods. These stimuli will be tested in a situation in which the patient will actively engage with a device that provides either a calming tactile stimulus without an odor, or one with an odor. These two groups will be compared to a control group that will not get a device with calming properties.

It is believed that the active engagement with the object and the self-motivated sensory exploration will have better outcomes than forced exposure to stimuli. Embedded within a culture of changing practices in psychiatry a brief and powerful study will be introduced to compare two different sensory stimuli to the treatment as usual.

Being in a Stressful Environment: The Problem of Anxious, Agitated, and Violent Psychiatric Patients

Arousal, anxiety, anger, aggression, and other acting out behaviors are unfortunately all too common among psychiatric patients in acute care and inpatient settings. Consequently, these behaviors constitute a complex safety issue. Patient-staff assaults have been most frequently studied, likely because staff is a more frequent target of assaultive behavior than other patients. Research indicates that among staff members, nurses are particularly vulnerable to attacks (Nolan et al., “Violence in Mental Health Care: The Experiences of Mental Health Nurses and Psychiatrists,” J. Adv. Nurs. 30:934-941 (1999), which is hereby incorporated by reference in its entirety), which likely is related to them often being the first responders to agitated patients, and also being the staff administering the intervention.

Among clinicians, psychiatrists are more likely to be targets of violence than other mental health care providers and reports indicate that more than a third of psychiatrists have been assaulted by a patient at least once and that the risk of violent victimization increases with less experience (see Gray, “Assaults by Patients Against Psychiatric Residents at a Public Psychiatric Hospital,” Academic Psychiatry 13:81-85 (1969); Ruben et al., “Physical Attacks on Psychiatric Residents by Patients,” J. Nerv. Ment. Dis. 168:243-245 (1980); Bernstein, “Survey of Threats and Assaults Directed Toward Psychotherapists,” Am. J. Psychother. 35:542-549 (1981); Black et al., “Assaults by Patients on Psychiatric Residents at Three Training Sites,” Hosp. Community Psychiatry 45:706-710 (1994); Schwartz et al., “Assaults by Patients on Psychiatric Residents: A Survey and Training Recommendations,” Psychiatr. Serv. 50:381-383 (1999), which are hereby incorporated by reference in their entirety): 73% to 96% of psychiatry residents have been verbally threatened and 36% to 56% have experienced physical assaults. Although few studies report on patient-patient assaults, it is clear that assaults occur more frequently among psychiatric patients than other medical patients (Blow et al., “Repeated Assaults by Patients in VA Hospital and Clinic Settings,” Psychiatr. Serv. 50:390-394 (1999), which is hereby incorporated by reference in its entirety).

Management of Challenging Patients

In the emergency room and inpatient settings, patients often have impaired cognition due to acute psychiatric states, substance intoxication/withdrawal, and/or over-arousal such that reasoning and conversation are not effective interventions, and situations rapidly escalate to restraint, seclusion, and involuntary medication for the safety of the patient, other patients, and the staff. There are a variety of methods for responding to and managing challenging patients who may display symptoms of arousal/stress, anxiety, agitation, and aggression. Such methods include: physical restraint, seclusion, pharmacological intervention, removal from area, transfer to other ward, and immediate verbal counseling. Unfortunately, few studies have empirically investigated these techniques. A study of 101 surveyed clinicians distilled three elements of effective responses to violent behavior: biological (physical or chemical restraints), psychological (verbal methods of de-escalation of the situation), and social (use of institutional, family, or peer influence) (Whitman et al., “Assault on the Therapist,” Am. J. Psychiatry 133:426-429 (1976), which is hereby incorporated by reference in its entirety). Another study highlighted the importance of staff training in nonviolent self-defense, restraint and seclusion procedures, alternatives to restraint and seclusion, identification of high-risk patients, improved security, and post-incident crisis counseling (Flannery et al., “Characteristics of Staff Victims of Psychiatric Patient Assaults: Updated Review of Findings,” Am. J. Alzheimer's Dis. Other Demen. 19:35-38 (2004), which is hereby incorporated by reference in its entirety).

Other suggested methods for deescalating agitated and threatening patients are implementing social norms against agitation and aggressive behavior within the patient/staff community that are maintained through periodic meetings, increasing staff awareness, and adherence to existing policies for the management of challenging patients, staff recognition of counter-transferential feelings related to assaultive patients, teaching trainees about the psychological domains of stress, agitation, and aggression, and placing written guidelines regarding safety issues in patients' charts where they can be followed up on by staff supervisors (see Black et al., “Assaults by Patients on Psychiatric Residents at Three Training Sites,” Hosp. Community Psychiatry 45:706-710 (1994), which is hereby incorporated by reference in its entirety).

Research focusing specifically on physical restraint and seclusion methods for containing challenging and threatening psychiatric patients is at best inconsistent (see Bower et al., “A Synthesis of What We Know About the Use of Physical Restraints and Seclusion with Patients in Psychiatric and Acute Care Settings: 2003 Update,” Online J. Knowl. Synth. Nurs. 10:1 (2003), which is hereby incorporated by reference in its entirety), and people in the field and in the public are becoming increasingly negative towards the use of such methods. This is illustrated by the increased focus of JCAHO to limit the use of physical restraints, seclusion, and the administration of deep sedation (see JCAHO Accreditation Process Guide for Hospitals 2009; Standards PC.03.01.03. to PC.03.03.31, which are hereby incorporated by reference in their entirety).

Adding to the problem is the concern that many mental health staff members have received inadequate training in managing aggressive and violent patients (Faulkner et al., “Threats and Assaults Against Psychiatrists,” Bull. Am. Acad. Psychiatry Law 18:37-46 (1990); Schwartz et al., “Assaults by Patients on Psychiatric Residents: A Survey and Training Recommendations,” Psychiatr. Serv. 50:381-383 (1999), which are hereby incorporated by reference in their entirety). Task forces from both the American Psychiatric Association (Dubin W, Lion J, (eds): Clinician Safety: Report of the American Psychiatric Association Task Force on Clinician Safety, Washington, D.C., American Psychiatric Press, 1993, which is hereby incorporate by reference in its entirety) and the American Psychological Association (APA 2000) have investigated the problem and recommended improving the training of Clinicians in the assessment and management of violent patients.

Sensory stimulation approaches may offer a new and simplified alternative to seclusion and physical restraint and involuntary medication in that limited staff training is necessary and patients are reducing arousal/stress and agitation by self-soothing.

Sensory Approaches to Reducing Arousal/Stress and Agitation in Other Populations

Scents and odors have long been known to decrease stress responsivity. The practice of aromatherapy is in part based on this belief. Most of the claims that have been made about the effects of odors on mood or behavior in humans have been based on anecdotal reports without empirical support. This should not distract from the fact that a few uses of odors in clinical settings have been scientifically tested and shown to be effective. One such area is the management of agitated behavior in patients with dementia (Ballard et al., “Aromatherapy as a Safe and Effective Treatment for the Management of Agitation in Severe Dementia: The Results of a Double-blind, Placebo-controlled Trial with Melissa,” J. Clin. Psychiatry 63(7):553-8 (2002); Holmes et al., “Lavender Oil as a Treatment for Agitated Behaviour in Severe Dementia: A Placebo Controlled Study,” Int. J. Geriatr. Psychiatry 17(4):305-8 (2002), which are hereby incorporated by reference in their entirety).

Strong Preliminary Data Driving the Study Hypotheses

Preliminary research shows that sniffing odors significantly lowered levels of salivary cortisol by 20% in healthy subjects (N=19). Cortisol was also lowered to a lesser degree in a control task during sniffing of the odorless solvent. This suggests that sensory engagement and exploration of odors may alone have a calming effect (the effect of “breathing” as a reducer of stress). The salivary cortisol levels in these experiments are consistent with self-reported mood that was measured before and after odor exposure. An effect of improving mood, however, was odor-specific to octyl acetate; an odor that smells similar to oranges. This is the odor proposed for use in the current study and the odor being used in the preliminary study.

Addressing the Scientific Knowledge Gap

Presently, there is a dearth of systematic research on the olfactory and tactile approaches to reducing over arousal/stress, anxiety, agitation, or aggressive behavior in psychiatric patients. Finding alternative methods for reducing stress and agitation in patients has become notably important after JCAHO has stipulated the reduction of use of seclusion and physical restraint methods. Most current interventions focus on verbal counseling. The present invention employs an innovative sensory stimulation approach that offers patients a tactile stimulus that can be infused with an odor.

Stress reduction in a highly challenging population (acute, forensic, and general psychiatric inpatients, adults, and children/adolescents) will be studied in a milieu well-known for its psychiatric services (Bellevue Hospital).

Scientific Opportunity

The clinical location of the study in the Bellevue Psychiatric hospital emergency room and inpatient units is a “real-world” setting to examine the efficacy of CAM to reduce episodes of restraint, seclusion, involuntary medications, and as needed medication doses. The current opportunity is to scientifically examine a novel approach, which adheres to the new JCAHO guidelines of reducing the use of restraint, seclusion, and administration of deep sedation.

The Ethnically Diverse and Underrepresented Research Population

This opportunity to test this device presents the opportunity to conduct innovative clinical research in a public sector hospital whose patients are among the most diverse in the country. Many of these patients have severe and persistent psychiatric illnesses, few resources, and receive treatment in the public sector. The focus of this research is on optimizing care for these people and finding an alternative to present treatment and management of challenging and threatening patients. Optimal treatment and management of severe and psychiatric patients will advance community health and wellness, decrease the prison population, and lead to enhanced stabilization of the American society.

Rigorous Design, High Impact

This research addresses the need for empirical studies on the efficacy of sensory stimuli (or CAM) for the use on stressed, agitated, and aggressive psychiatric patients. This research will be maximally informative, using both objective endpoints and salivary cortisol as a biomarker of stress response reduction. This research attends to a range of endpoints meaningful to improved health of mentally ill people and the safety of the hospital setting. The results may have a high impact in the biomedical care of threatening and imminent behavioral conditions.

The scientific discoveries anticipated are likely to impact the economy positively by improving the productivity of the US workforce by decreasing time off among nursing, clinical, and other staff due to patient assaults. In addition, staff will be able to dedicate more time to other patients requiring their aid. Simultaneously, by using an alternative to sedative medication, the scientific discovery of efficacious sensory stimuli will also reduce the nation's overall healthcare expenditure on pharmacological treatment.

### Prophetic Example 3

### The Approach

The research will test the device and method of the present invention in Bellevue Hospital's psychiatric emergency room and on inpatient civilian and forensic psychiatric units. It will compare the effectiveness of two different Complementary Alternative Medicine (CAM) approaches that will be used in addition to standard conventional care versus standard conventional care alone in the inpatient psychiatric settings at Bellevue Hospital in New York City.

Setting: Bellevue Hospital Center (New York University School of Medicine)

One of the largest single municipal hospital buildings in North America and the flagship facility of New York City's Health and Hospitals Corporation, The Department of Psychiatry at Bellevue Hospital is the best known department of psychiatry in the country. It is widely considered to be the “birthplace of American Psychiatry.” It is the oldest public hospital in the country, having served patients since 1736. Today's Bellevue is a modern, full-service facility providing comprehensive medical, psychiatric, and social services, including inpatient, outpatient and world-renowned emergency care.

Nearly half of all the beds at Bellevue are in the Department of Psychiatry. The Department of Psychiatry at Bellevue Hospital operates 359 beds; approximately 8,000 emergency room visits; 170,000 outpatient visits; and 7,000 consultations a year. The Department of Psychiatry at Bellevue Hospital is directed by Dr. Mary Ann Badaracco. It is organized into six divisions: Forensic Psychiatry, Emergency Psychiatry, Consultation Liaison (Psychosomatic), Ambulatory and Community Psychiatry, Substance Abuse, Geriatric Psychiatry, and Adult In-Patient Psychiatry. It also has a vigorous Child and Adolescent Psychiatry department, directed by Jennifer Havens. Over the last few years, the psychiatry program has achieved national and international recognition for its outstanding quality of care. The JCAHO has certified their programs at 100% compliance in the last three surveys. Bellevue Hospital is also the only public psychiatry hospital to be ranked by the US News and World Report as among the best 25 in the nation. Eighty percent of Bellevue's patients come from the city's medically underserved populations.

Relevant Hospital Units for This Research

The Comprehensive Psychiatric Emergency Room (CPEP) is the busiest psychiatric emergency service in New York City with approximately 8,000 emergency room visits per year.

There are 14 Inpatient Psychiatric Units, housing 20-30 patients at a given time for an average of 23 days. Two units will be dedicated to the study.

There are 2 Inpatient Child and Adolescent Units, each 15-bed units housing children and adolescents under 18 years of age.

The Forensic Inpatient Psychiatry Service is a two-unit, 68-bed inpatient service that provides acute psychiatric care to adult male jail inmates from New York City. One unit will be dedicated to the study.

Current Use of Seclusion, Physical Restraints, and Medication Intervention for Challenging and Threatening Patients at Bellevue Hospital

Over the past two years there has been an increase in patient-patient and patient-staff assaults at Bellevue Hospital that has paralleled a decrease in restraint and seclusion, and use of involuntary IM medications. There has not been an increase in the administration of oral medications or other, non-coercive measures (behavioral, verbal, sensory) to reduce escalating arousal/stress and agitation among psychiatric patients.

Estimates of 2008 Statistics for Restraints, Seclusion, Involuntary Medications, and Oral Medication at Bellevue Hospital

CPEP (Psychiatric ER)

Restraint/seclusion: 2.2% of 8400 presentations to CPEP. 184 total episodes.

Involuntary medications: 8.2% of 8400 presentations. 691 total episodes.

Oral medications: 1200-1500 episodes of oral medication given annually, or 100-125 per month.

Inpatient Units

Restraint/seclusion: 63 total episodes in 2008, which includes all inpatient units.

Involuntary medications: ˜15 episodes per month, per unit, with an average census of 27 patients.

Oral medications: 30-50 episodes per month, per unit, with same average census. 360-600 annually.

These data show the importance of bringing complementary and alternative treatments to the interface of treating over aroused, hostile, and potentially aggressive patients. The goal is to reduce physical altercations to zero incidents.

### Prophetic Example 4

### Method

This method is for a large and methodologically rigorous program of study to compare the effectiveness of treatment as usual with two novel interventions, tactile-only and tactile-smell, in reducing episodes of restraint, seclusion, and involuntary medication.

Subjects and Recruitment

A total of 700 psychiatric patients will be enrolled: 600 adult psychiatric patients (300 acute psychiatric patients from the ER; 200 inpatients; 100 forensic psychiatric inpatients) who are >18 years old and have the capacity to give informed written consent and do so freely, as well as 100 children or adolescents, <18 years old, who provide parental or legal guardian assent. The adult subjects will be recruited from the acute psychiatric inpatient unit, three regular psychiatric inpatient wards, and two forensic psychiatric units, and the children and adolescents will be recruited from the child and adolescent psychiatry inpatient units. All subjects will be recruited from Bellevue Hospital, which operates under the clinical responsibility of New York University School of Medicine. Inpatients and cases presenting to the ER will be invited to sign informed consent for the study when they are admitted to the unit or when they arrive at the ER. Subject Flow will be abundant, easily yielding the subjects from which to recruit.

With support from the American Psychological Foundation, data is currently being collected on a preliminary model of the device of the present invention on the psychiatric inpatient units (adults).

Study participants will give permission in their consent form to be offered a device of the present invention when they are escalated or observed to be escalating by clinical staff. The patients will be asked to take the item, hold it (they will be able to manipulate it/play with it), and try to “calm down.” In condition 1, no device of the present invention will be given. In condition 2, it will be a tactile-only device. In condition 3, it will be a tactile-smell device of the present invention. Brief clinical ratings, patient reported distress, and a salivary sample will be obtained before the device is given, after 15 minutes while holding the device, and 30 minutes upon returning the device.

Double Blind Intervention with Respect to the Scent

Neither the staff that administer the device, nor the patients, will be aware of the study blind. Upon admission, interested patients will be explained the purpose of the study, and, more specifically, be explained that in the case of feeling stressed or agitated they will be offered the sensory stimulation device, that may have a tactile and olfactory calming effect in addition to standard treatment. Whether or not the object has been scented will be according to a predetermined schedule of conditions. As part of the consent form, all subjects will also be explained that they can terminate participation in the study at any time without affecting their regular treatment or refuse the device at any time.

Scent

Octyl acetate (CH₃COOC₈H₁₇) will be used for the smell. It is a colorless clear liquid that is readily available at 98 to 100% purity and it is commonly used as an artificial odor in many foods and household items. It is an ester, which can be made artificially from octanol (octyl alcohol) and acetic acid. It is the basis of artificial orange flavoring. Its odor type is floral; its odor strength is medium. It has been described as orange, earthy, mushroom, herbal, and waxy. It has a long shelf life of 24 month(s) or longer if stored properly in a cool dry place in tightly sealed containers, protected from heat and light. It is insoluble in water.

A preliminary study shows that this odor lowers cortisol levels in healthy subjects.

Salivary Cortisol

Before the device is handed to the patient, a salivary sample is obtained by letting the patient chew on a lemon-scented cotton ball that is designed for collecting saliva (Salivette, Sarstedt). Another sample is obtained after the patient's interaction with the multisensory device and a final sample will be collected 30 minutes later. The analyses of the cortisol in the saliva will be carried out by NYU Core laboratory (part of the NYU Clinical Translational Science Institute), and the changes in cortisol between the samples will be calculated.

Data on Diagnosis and Demographic Measures for All Subjects Who Sign Informed Consent

Clinical information and diagnosis will be ascertained. A current and best estimate lifetime diagnosis will be generated from the information. Also, demographic information (race, ethnicity, gender, education, housing, occupation, social network) will be obtained.

Data on Episodes

Subjects receiving the intervention will also be assessed on clinical measures and a biomarker of arousal and agitation (salivary cortisol) measured at three time points (before presenting device after 15 minutes while holding the device, and 30 minutes after returning the device).

Salivary samples will only be collected for one episode per patient. However, for subjects who have more than one episode and want to de-escalate holding a device, additional devices will be available. Clinical measures will be collected on the additional episodes.

Symptom Assessments Before, During, and After Administration

By Subject

A brief questionnaire designed to be completed in less than 1 minute is used to rate anger, fear, anxiety, stress, and sadness on a Likert-scale.

By Staff

Agitation and Aggression: The Overt Aggression Scale (OAS; Yudofsky et al., “The Overt Aggression Scale for the Objective Rating of Verbal and Physical Aggression,” Am. J. Psychiatry 143:35-39 (1986), which is hereby incorporated by reference in its entirety), designed to assess observable aggressive or violent behavior (verbal aggression, physical aggression, physical aggression against self, and physical aggression against other people) rated according to severity.

Physiological Stress

Cortisol Level: The salivary sample for the measurement of cortisol level is obtained by letting the patient chew on a lemon-scented cotton ball specifically designed for collecting saliva (Salivette, Sarstedt).

Gender Effects

The research group has deep and broad experience in all aspects of the human subject research and protection. The study adds a unique aspect to gender differences research by focusing on an innovative sensory stimulation object with potential gender biased advantages in reducing stress and agitation in psychiatric patients.

Assessment Team

All chart abstractors/interviewers have experience in mental health treatment at a BA or BS level or above. Interviewers are trained to maintain a reliability criterion of at least 0.70 (reliability coefficients calculated for 20% of the sample). Interviewers will consist of trained staff. Research assistants will collect research diagnoses and other demographic information from chart information.

Credentials of the Research Team for Launching Successful Research Programs

This is an experienced multidisciplinary research group with existing collaborative ties and integrated expertise in forensic and emergency psychiatry, patient-oriented research, and basic olfactory neuroscience. The scientific environment, institutional support, equipment, and other physical resources needed for the research are available to the investigators. The setting of a large public hospital (Bellevue Hospital) on the campus of the strong and supportive academic environment of the NYU Langone Medical Center provides an exceptional opportunity to conduct research on a challenging problem for an underserved population. The team includes beginning investigators and an accomplished mentor of beginning clinical investigators.

### Prophetic Example 5

### Data Analysis

This study is a clinical trial to examine the efficacy of using a sensory method/intervention to reduce patient “tension” at a time when his/her clinical condition is escalating. The plan is to recruit a total of seven hundred patients that will sign informed consent documents to enter the study at the time that they enter the ER or are admitted to an inpatient unit.

It is estimated that 50% of these 700 participants will have an episode of escalating or current agitation that will reach clinical attention and be sufficient for an “as needed” clinical intervention. Thus, of the 700 recruited cases, 350 will have an episode in which they receive a device described herein as intervention, 175 will receive the tactile-only device and 175 will receive the tactile-smell device of the present invention. The current study will compare the two complementary self-soothing items to treatment as usual. The soothing items will be scented or unscented sensory stimuli items delivered in a double blind fashion.

During data analysis, the first step will be to examine the descriptive statistics (means and standard deviations) and distributions of all measures whether categorical or continuous to identify key features (non-normal distribution, outliers, skewness) that might impact inferential methods. If the distribution of variables exhibits skewness or other marked departure from normality, appropriate transformations will be applied or non-parametric methods will be used for analysis. The demographic characteristics will be examined carefully in association with the study conditions using chi-square for categorical variables and ANOVA for continuous variables. If any of these characteristics prove to be significantly different, they will be adjusted for by including them as covariates or factors during subsequent analyses.

Hypothesis Testing

Three hypotheses are posited and their analyses are addressed here.

Hypothesis 1

Cases with the tactile-smell device of the present invention will have significantly fewer episodes of restraint/seclusion/involuntary medication than those who receive the tactile-only device. Furthermore, both groups receiving the devices of the present invention (with or without smell) will have significantly fewer episodes of restraint/seclusion/involuntary medication than those who receive no device (treatment as usual).

This hypothesis will be addressed using the Chi-square statistics comparing the number of patients experiencing an episode across the three conditions, and then post-hoc across the two device conditions.

Hypothesis 2

Cases with the tactile-smell device will have significantly greater declines in clinical ratings and salivary cortisol than those who receive the tactile-only device.

When an episode seems likely to occur, a previously consented patient will be assessed for salivary cortisol, aggression scale rating (OAS) and self report (Likert scale) estimates of anger, fear, anxiety, stress, and sadness. A device representing two of the conditions (device with or without smell) will be given to the patient, or no device will be given to the patient. Follow-up assessments of salivary cortisol, the aggression scale rating, and the self report estimates of anger, fear, anxiety, stress, and sadness will be repeated at approximately 15 and 30 minutes for patients in all 3 conditions. However, patients not receiving a device may have to receive a usual intervention (restraint/seclusion, involuntary medication, etc.) that may deem their follow-up disputable.

This hypothesis will be addressed using a GEE (general estimating equations: an extension of the general linear model to allow for the analysis of repeated measures or other correlated observations) procedure to assess differences between the conditions across the 3 assessment points, with the inclusion of appropriate covariates such as gender, age, housing situation, and employment status.

Hypothesis 3

During the study month blocks (device conditions) there will be fewer restraint, seclusion, and involuntary medication episodes overall than during the non-study (treatment as usual) month blocks in each setting. Secondarily, the ameliorative effect will be greater for the study month blocks with the device with odor (tactile with odor) than for the blocks with the device without odor (tactile-only).

This hypothesis will be addressed using the Chi-square statistics comparing the number of patients experiencing an episode across conditions.

Additional Hypotheses to be Examined

Ecological Indicators across All Study Settings of Complementary Study versus Regular Clinical Care

The 700 hundred cases recruited into this study will only represent a fraction of the total number of client patients on the various psychiatric units involved. The number of episodes (assault, involuntary medication, restraint, or seclusion) across all 5 settings will be determined in each 3-month epoch, accounting for a total census.

It is hypothesized that there will be fewer episodes (assault, involuntary medication, restraint, or seclusion) during the device study months than during treatment as usual and that the tactile-smell device will have a greater reduction on such episodes than the tactile-only intervention.

Individual Qualitative Data

For those receiving the device intervention, the scented and unscented device interventions will be compared for effects on clinical treatment, including whether the patient went on to receive involuntary medication, restraint, or seclusion. The nursing notes for the preceding and subsequent 24 hours will be abstracted.

Predicting Episodes

Chart review diagnostic procedures and demographic information will be obtained on all 700 enrolling cases to examine the predictors of clinical escalation among the 700 enrolled patients.

Power Considerations

For Hypotheses 1 and 3, the null hypothesis is that the rates for the occurrence of episodes of assaults, restraint/seclusion, involuntary medication, or the need to be medicated are equal across conditions. Given an alpha set at 0.05 for significance and the test is two-tailed; with the proposed sample sizes of 175 for the device conditions and 350 for the non-device condition, a rate difference of 0.13-0.14 (13-14%, [i.e., 0.37 versus 0.50]) between conditions will yield power=0.80.

Related to the salivary cortisol measurements of Hypothesis 2, salivary cortisol measurements were measured before and after a smell stimulus (same odor intended to use) and showed on average 20% decrease, representing an effect size=0.67 (medium-large as per Cohen's conventions). Assuming a similar effect size, power exceeds 0.80. Additional power calculations indicate that with all condition Ns exceeding 175 cases, it would exceed power=0.80 with an effect size as small as=0.25. This would also be true for the Overt Aggression Scale and the self report (Likert scales) estimates of anger, fear, anxiety, stress, and sadness.

Unique Window of Opportunity for this Proposal to Address Questions in the Field

Many CAM methods will soon be implemented across the United States. JCAHO has stipulated that seclusion and restraint methods to contain agitated patients must become a “last resort.” However, CAM methods lack empirical validation in this setting, as no scientifically rigorous trials have been conducted in a busy inner-city hospital's inpatient and emergency psychiatric department.

The proposed research can provide evidence based information on the implementation of CAM in the setting of psychiatric escalation in the hospital setting.

Although the invention has been described in detail for the purposes of illustration, it is understood that such detail is solely for that purpose, and variations can be made therein by those skilled in the art without departing from the spirit and scope of the invention which is defined by the following claims.

What is claimed:

1. A sensory device for reducing tension in a subject comprising:
an elastic covering;
a filling material inside the elastic covering;
an openable sealed reservoir located within the filling;
an absorbent material surrounding the reservoir; and
a calming odor releasing substance sealed within the reservoir.

2. The device of claim 1, wherein the odor releasing substance comprises octyl acetate.

3. The device of claim 1, wherein the odor releasing substance comprises about 5 ml of an about 1/100 dilution of octyl acetate.

4. The device of claim 1, wherein the device is oval shaped, about 40 cm in length and about 20 cm in diameter.

5. The device of claim 1, wherein the elastic covering comprises about 84% nylon and about 16% spandex.

6. The device of claim 1, wherein the reservoir is a sealed breakable ampoule.

7. The device of claim 6, wherein the ampoule is made of plastic.

8. The device of claim 1, wherein the absorbent material comprises nonwoven polypropylene.

9. The device of claim 1, wherein the filling material comprises porous silicone or polystyrene foam beans.

10. The device of claim 1, wherein the covering comprises a textured silicone surface.

11. A method of reducing tension in a subject, said method comprising:
providing a subject in need of tension relief with the device of claim 1;
manipulating the device to break open the reservoir and release the calming odor releasing substance; and
inhaling the released calming odor in an amount sufficient to lower salivary cortisol levels in the subject and thereby reduce tension in the subject.

12. The method of claim 11, wherein the subject is a human subject.

13. The method of claim 11, wherein the subject is exhibiting stress, agitation, aggression, and/or violence.

14. The method of claim 11, wherein said manipulating is carried out by squeezing the device.

15. The method of claim 11, wherein the odor releasing substance comprises octyl acetate.

16. The method of claim 11, wherein the odor releasing substance comprises about 5 ml of an about 1/100 dilution of octyl acetate.

17. The method of claim 11, wherein the device is oval shaped, about 40 cm in length and about 20 cm in diameter.

18. The method of claim 11, wherein the elastic covering comprises about 84% nylon and about 16% spandex.

19. The method of claim 11, wherein the reservoir is a sealed breakable ampoule.

20. The method of claim 19, wherein the ampoule is made of plastic.

21. The method of claim 11, wherein the absorbent material comprises nonwoven polypropylene.

22. The method of claim 11, wherein the filling material comprises porous silicone or polystyrene foam beans.

23. The method of claim 11, wherein the covering comprises a textured silicone surface.
